(19) United States
(12) Patent Application Publication
(10) Pub. No.: US 2026/0108309 A1
(43) Pub. Date: Apr. 23, 2026

(54) DETERMINATION METHOD AND DETERMINATION APPARATUS FOR UNDER-NAVIGATION APICAL SURGERY PATH

(71) Applicant: WUHAN UNIVERSITY, Hubei (CN)

(72) Inventors: Liuyan MENG, Hubei (CN); Zhuan BIAN, Hubei (CN); Yun CHEN, Hubei (CN); Chen CHEN, Hubei (CN); Li QIN, Hubei (CN); Yan TAO, Hubei (CN); Yeyu LIN, Hubei (CN)

(73) Assignee: WUHAN UNIVERSITY, Hubei (CN)

(21) Appl. No.: 19/303,295

(22) Filed: Aug. 18, 2025

(30) Foreign Application Priority Data
Oct. 23, 2024 (CN) 202411479259.9

(51) Int. Cl. A61B 34/20; A61B 6/51; A61B 34/10; A61B 90/00; A61C 5/40

(52) U.S. Cl. CPC A61B 34/20; A61B 6/51; A61C 5/40; A61B 2034/107; A61B 2034/2065; A61B 2090/3762

(57) ABSTRACT

A determining method for an under-navigation apical surgery path is provided. The determining method includes the following steps: S1, obtaining CT image data of affected teeth and jaw bone data of a patient, and calculating the CT image data of the affected teeth to obtain calculation parameters of the surgery path; S2, based on the calculation parameters of the surgery path and in combination with the data of the affected teeth and the jaw bone data, obtaining a start point and an ending point of the surgery path, and generating the surgery path based on the start point and the ending point.

### CROSS-REFERENCE TO RELATED APPLICATION

This application claims the priority benefit of China application no. 202411479259.9, filed on Oct. 23, 2024. The entirety of the above-mentioned patent application is hereby incorporated by reference herein and made a part of this specification.

### TECHNICAL FIELD

The present disclosure relates to the technical field of medical navigation, and in particular to a determination method and determination apparatus for under-navigation apical surgery path.

### DESCRIPTION OF RELATED ART

When the conventional root canal treatment or root canal retreatment cannot control the infection of the periapical tissues, apical microsurgery can be used as an effective treatment approach. With cutting the root tip 3 mm in a direction perpendicular to dental long axis as basic principle, the success rate is as high as 95%. However, it is always a big difficulty in apical microsurgery field to accurately locate the root tip and carry out accurate minimally-invasive osteotomy and apicectomy. In recent years, the application of the dynamic navigation technology in the apical surgeries has been widely recognized. The current pre-dynamic navigation surgery design requires the pre-surgery CT image data of the patients to be exported into a dedicated design software to realize three-dimensional reconstruction for the surgical area of the patients. Then, the doctors can carry out detailed pre-surgical planning for the surgery path using three-dimensional image information in the design software so as to complete the design of the surgery plan. But, the plan design, to some extent, depends on the professional skills and experience of the patients. The doctors need to perform complex operations using the CT images in the professional software environment, requiring a long learning curve and high reliance on the individual experience. In order to improve the design accuracy, it is usually required to perform manual adjustment and calibration repeatedly, leading to a long tedious process. Therefore, although the dynamic navigation technology provides an importance assistance for the apical microsurgery, the accuracy and efficiency of the surgery plan design remain to be further optimized and improved.

### SUMMARY

In order to address the shortcomings of the prior arts, the present disclosure provides a determination method of an under-navigation apical surgery path, which improves the pre-surgical design efficiency, reduces the learning costs of the doctors and standardizes plan design with high accuracy.

In order to solve the above technical problems, the present disclosure employs the following technical solution.

A determination method of an under-navigation apical surgery path, comprising the following steps:
at step S1, obtaining CT image data of affected teeth and jaw bone data of a patient, and calculating the CT image data of the affected teeth to obtain calculation parameters of the surgery path;
at step S2, based on the calculation parameters of the surgery path and in combination with the data of the affected teeth and the jaw bone data, obtaining a start point and an ending point of the surgery path, and generating the surgery path based on the start point and the ending point;
wherein the calculation parameters of the step S1 comprise a dental root tip point root_tip and a direction root_vec of a dental root long axis;

Furthermore, the method of obtaining the calculation parameters comprises:
at step S101, obtaining three-dimensional model data of the affected teeth based on the CT image of the affected teeth to obtain a three-dimensional model of the affected teeth, and by using computer graphics algorithm, obtaining a center root_center of polydata of a dental root in the three-dimensional model of the affected teeth, wherein polydata refers to three-dimensional model data of the dental root;
at step S102, taking a center root_center of the dental root as origin and taking a direction of a dental long axis as normal direction, making a root-end resection plane plane_cut, wherein the direction of the dental long axis refers to pointing from a dental crown toward a dental root;
at step S103, by using the root-end resection plane plane_cut, cutting the three-dimensional model of the affected teeth into a first part part1 and a second part part2, using the first part part1 to calculate a connected region to obtain n connected regions and extracting root_polydata of a single connected region, wherein root_polydata refers to root three-dimensional data of a single connected region; and calculating the dental root tip point root_tip based on the root three-dimensional data root_polydata of the single connected region;
wherein the first part part1 is a positive part of the root-end resection plane plane_cut, and the second part part 2 is a negative part of the root-end resection plane plane_cut; the dental root tip point root_tip is an angular point closest to a long axis origin in all angular points on the root_polydata projected to the dental long axis;
at step S104, calculating a length left_length of a line connecting an origin orgin_cut on the root-end resection plane plane_cut with the dental root tip point root_tip, and determining whether the length left_length of the connection line exceeds a threshold; when the length left length of the connection line exceeds the threshold, step S105 is performed; when the length left_length does not exceed the threshold, step S106 is performed;
at step S105, using a new origin obtained by translating the origin orgin_cut by a distance along the direction of the dental long axis, and using the direction of the dental long axis as a normal direction, making the root-end resection plane plane_cut again, and repeating the steps S103 to S104 to perform multiple iterations on the single connected regions to extract the most connected regions until the length left_length is not beyond the threshold;
at step S106, obtaining a second root-end resection plane by translating the root-end resection plane by a distance along the normal direction, and using the second root-end resection plane to cut the root_polydata into a first part part1′ and a second part part2′ again, wherein the first part part1′ is a positive part of the second root-end resection plane and the second part part2′ is a negative part of the second root-end resection plane;
at step S107, using a center of the second part part2′ as a dental root top center root_top, using a line connecting the dental root tip point root_tip and the top center root_top as the direction root vec of the dental root long axis, and using the dental root tip point root_tip and the direction root vec of the dental root long axis as calculation parameters of the path.

Furthermore, by using computer graphics algorithm, obtaining the center root_center of the polydata of the dental root includes: by using computer graphics algorithm, segmenting the three-dimensional model of the affected teeth into a three-dimensional model of the dental crown and a three-dimensional model of the dental root, and calculating a centroid of the three-dimensional model of the dental root to obtain the center root_cente of the polydata of the dental root.

Furthermore, the method of performing the step S2 includes:
at step S201, moving the dental root tip point root_tip along the direction root_vec of the dental root long axis by an apicectomy distance to obtain a first calibration point Point1;
at step S202, making a first line segment Line1 through the first calibration point Point1 along a negative direction of a dental buccolingual direction Vec_coronal, and dividing intersection points of the first line segment Line1 and the three-dimensional model data polydata of the dental root into penetration-in points and penetration-out points;
wherein the penetration-in points and the penetration-out points are obtained by ranking the intersection points of the first line segment Line1 and the three-dimensional model data polydata of the dental root, wherein the odd-numbered points are penetration-in points and the even-numbered points are penetration-out points;
at step S203, finding the penetration-in point Point_tmp and the penetration-out point Point_end which are closest to the dental root tip point root_tip;
at step S204, forming intersection points obtained by the first line segment Line1 and the jaw bone data ; if the intersection points exist, taking a point among the intersection points, which is the closest point to a start point of the first line segment, as a start point Point_start of the path, and otherwise, taking the penetration-in point Point_tmp as the start point Point_start of the path.

Another object of the present disclosure is to provide a determination apparatus for performing the above under-navigation apical surgery path, which includes:
a data obtaining module, configured to obtain CT image data of affected teeth and jaw bone data of a patient;
a parameter calculating module, configured to calculate calculation parameters of the surgery path based on the CT image data of the affected teeth obtained by the data obtaining module;
a path generating module, configured to obtain a start point and an ending point of the surgery path based on the calculation parameters of the surgery path and the jaw bone data to generate the surgery path.

The present disclosure further provides an electronic apparatus, comprising a memory, and a processor, and the memory stores computer programs. The processor executes the computer programs to perform the steps of the above method.

The present disclosure further provides a computer readable storage medium, storing computer programs. The computer programs are executed by a processor to perform the steps of the above method.

Compared with the prior arts, the present disclosure has the beneficial effects: in the present disclosure, by performing three-dimensional patch formation on the dental CT data in the computer software, three-dimensional model data of single tooth is obtained; based on computer graphics algorithm, the three-dimensional model data is processed; in combination with the requirements of the clinical medicine for the apical surgery, an optimal path is recommended on the three-dimensional model. The present disclosure improves the pre-surgical design efficiency, reduces the learning costs of the doctors and changes the design plan into standard flow, avoiding the problems of the plan design accuracy depending on human factors, and increasing the plan accuracy.

### BRIEF DESCRIPTION OF THE DRAWINGS

FIG. 1 is a flowchart illustrating a determination method of an under-navigation apical surgery path according to an embodiment of the present disclosure.

FIG. 2 is a flowchart of obtaining path calculation parameters according to an embodiment of the present disclosure.

FIG. 3 is a flowchart of obtaining and designing a start point and an ending point of the path according to an embodiment of the present disclosure.

FIG. 4 is the CT images for demonstrating how to obtain the path according to an embodiment of the present disclosure.

### DESCRIPTION OF THE EMBODIMENTS

The technical solutions of the embodiments of the present disclosure will be clearly and fully described below in combination with the embodiments of the present disclosure. Apparently, the embodiments described hereunder are merely some embodiments of the present disclosure rather than all embodiments. All other embodiments obtained by those skilled in the arts based on these embodiments of the present disclosure without carrying out creative work shall fall within the scope of protection of the present disclosure.

It should be noted that, without conflicts, the embodiments in the present disclosure and the features in the embodiments can be mutually combined.

Further descriptions are made below to the present disclosure in combination with the specific embodiments but shall not constitute limitation to the present disclosure.

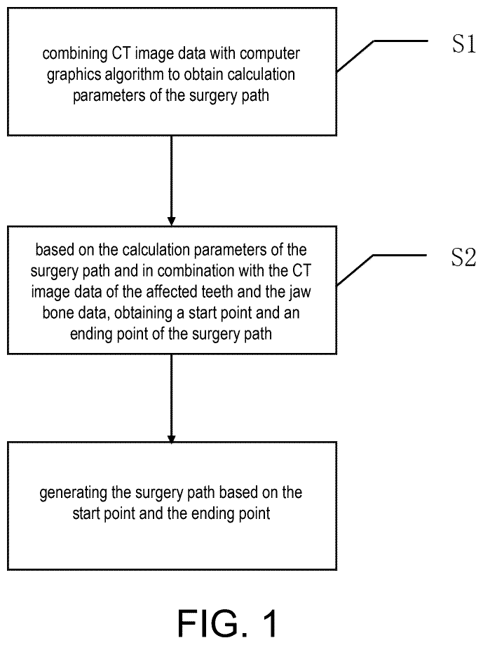

As shown in FIG. 1, an embodiment of the present disclosure provides a determination method of an under-navigation apical surgery path, which includes the following steps.

At step S1, CT image data of affected teeth and jaw bone data of a patient are obtained, and the CT image data of the affected teeth is calculated to obtain calculation parameters of the surgery path.

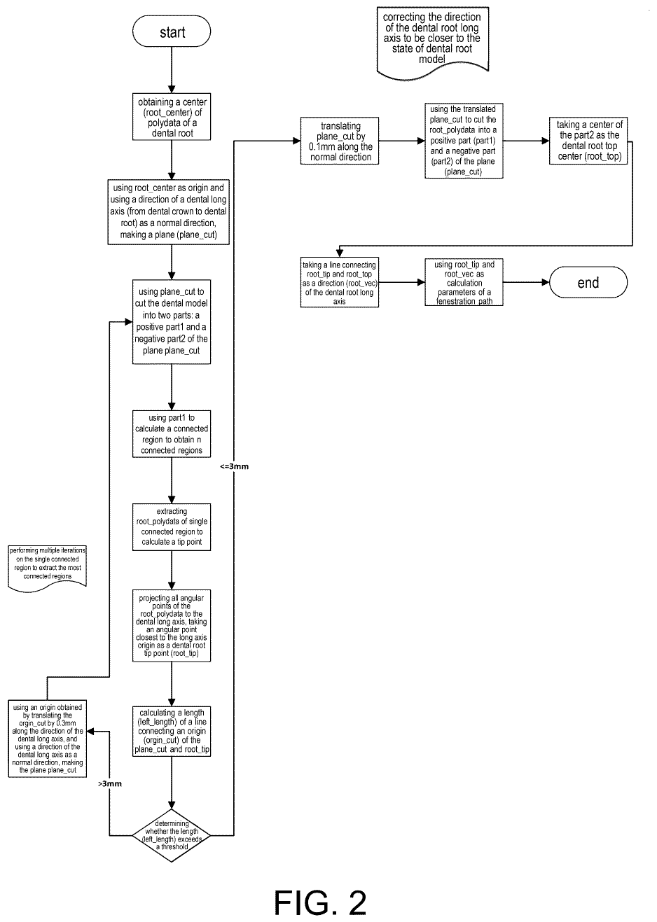

With reference to FIG. 2, the specific method of the step includes the following steps.

At step S101, in a software, three-dimensional patch formation is performed on the CT medical image data of the affected teeth to obtain three-dimensional model data of the affected teeth, and a three-dimensional model of the affected teeth is obtained based on the three-dimensional model data of single tooth; then by using computer graphics algorithm, the three-dimensional model of the affected teeth is segmented into a three-dimensional model of the dental crown and a three-dimensional model of the dental root, and a centroid of the three-dimensional model of the dental root is calculated to obtain a center root_center of polydata of a dental root, wherein polydata refers to three-dimensional model data of the dental root.

At step S102, taking a center root_center of the dental root as origin and taking a direction of a dental long axis as normal direction, a root-end resection plane plane_cut is made.

wherein the dental long axis refers to an imaginary line through the dental center, and the direction of the dental long axis refers to pointing from a dental crown toward a dental root.

At step S103, by using the root-end resection plane plane_cut, the three-dimensional model of the affected teeth is cut into a first part part1 and a second part part2; the first part part1 is used to calculate a connected region. When there are multiple dental roots, each dental root in the first part partlis possibly cut to form an independent three-dimension body (connected region of three-dimensional model of single dental root), and multiple dental roots are possibly fused into a large independent three-dimensional body (connected regions of three-dimensional models of multiple dental roots); each three-dimensional body is extracted to obtain n connected regions; root_polydata of single connected region is extracted and the dental root tip point root_tip is calculated based on the root_polydata of single connected region, where the root_polydata is root three-dimensional data of a single connected region.

The first part part1 is a positive part of the root-end resection plane plane_cut, and the second part part 2 is a negative part of the root-end resection plane plane_cut; the dental root tip point root_tip is an angular point closest to a long axis origin in all angular points on the root_polydata projected to the dental long axis.

At step S104, a length left_length of a line connecting an origin orgin_cut on the root-end resection plane plane_cut with the dental root tip point root_tip is calculated, and whether the length left_length exceeds a threshold is determined; when the length left_length exceeds the threshold, step S105 is performed; when the length left_length does not exceed the threshold, step S106 is performed.

In this embodiment, the threshold is set to a standard length 3 mm for apicectomy, and set based on actual situations of the affected teeth in other embodiments.

At S105, using a new origin obtained by translating the origin orgin_cut by 0.3 mm along the direction of the dental long axis, and using the direction of the dental long axis as a normal direction, the root-end resection plane plane_cut is made again, and the steps S103 to S104 are repeated until the length left_length is not beyond the threshold; thus, multiple iterations are performed on single connected region to extract the most connected regions, and further the multiple dental roots fused together are segmented out as possible.

At step S106, a second root-end resection plane is obtained by translating the root-end resection plane by a distance along the normal direction, where in this embodiment, the distance can be 0.1 mm or set based on tooth size; the direction of the dental root long axis is corrected to be closer to the state of the three-dimensional model of the dental root; the second root-end resection plane is used to cut the three-dimensional model data root_polydata of the dental root of the single connected region into a first part part1′ and a second part part2′ again.

The first part part1′ is a positive part of the second root-end resection plane and the second part part2′ is a negative part of the second root-end resection plane.

At step S107, a center of the second part part2′ obtained in the step S106 is used as a dental root top center root_top, a line connecting the dental root tip point root_tip and the dental root top center root_top is used as the direction root_vec of the dental root long axis, and the dental root tip point root_tip and the direction root_vec of the dental root long axis are used as calculation parameters of the path.

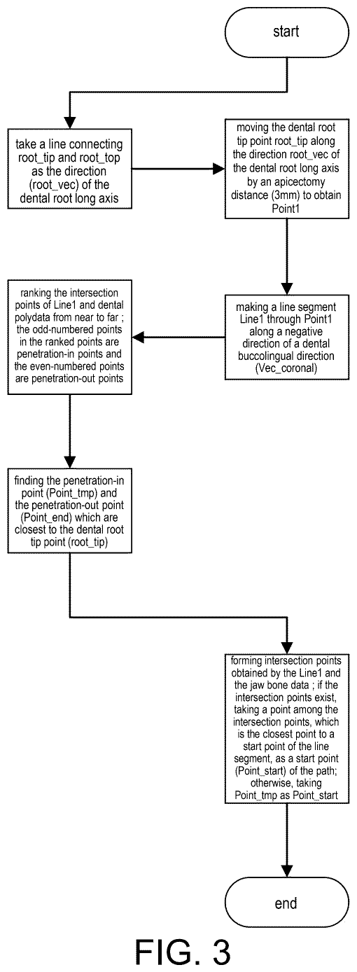

At step S2, based on the calculation parameters of the surgery path and in combination with the data of the affected teeth and the jaw bone data, a start point and an ending point of the surgery path are obtained, and the surgery path is generated based on the start point and the ending point as shown in FIG. 3. The step can be specifically performed in the following steps.

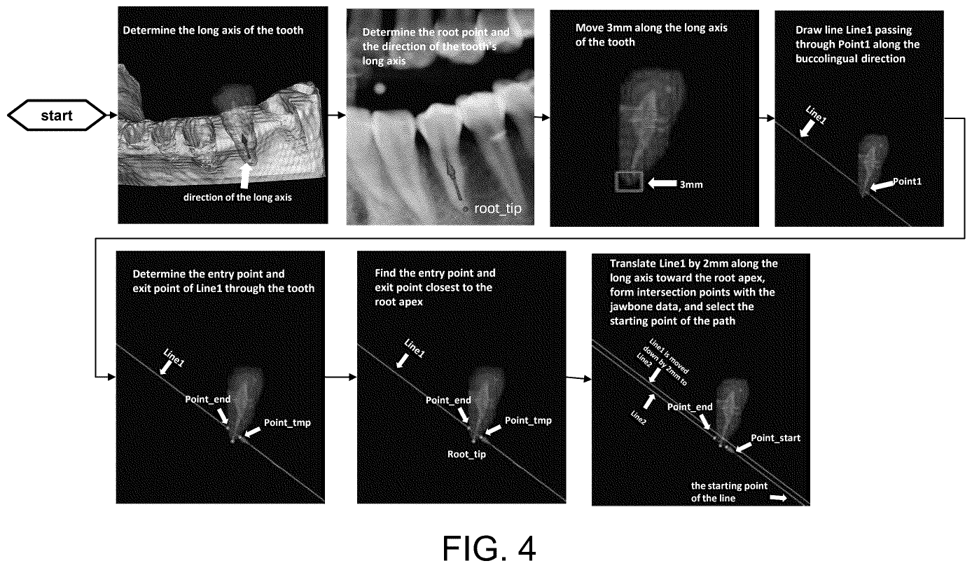

At step S201, the dental root tip point root_tip is moved along the direction root_vec of the dental root long axis by an apicectomy distance to obtain a first calibration point Point1. In this embodiment, the apicectomy distance is 3 mm (shown in FIG. 4) or set based on tooth size.

At step S202, along a negative direction of a dental buccolingual direction Vec_coronal, a first line segment Line1 through the first calibration point Point1 is made, and intersection points of the first line segment Line1 and the three-dimensional model data polydata of the dental root are divided into penetration-in points and penetration-out points;
wherein the penetration-in points and the penetration-out points are obtained by ranking the intersection points of the first line segment Line1 and the three-dimensional model data polydata of the dental root, where the odd-numbered points are penetration-in points and the even-numbered points are penetration-out points.

At step S203, the penetration-in point Point_tmp and the penetration-out point Point_end which are closest to the dental root tip point root_tip are found.

At step S204, if there are intersection points obtained by the first line segment Line1 and the jaw bone data, a point among the intersection points, which is the closest point to a start point of the first line segment, is taken as a start point Point_start of the path, and otherwise, the penetration-in point Point_tmp is taken as the start point Point_start of the path. The surgery path is generated in combination with the start point Point_start of the path and the penetration-out point Point_end, as shown in FIG. 4.

In one embodiment, there is provided a determination apparatus for the under-navigation apical surgery path, which includes:
a data obtaining module, configured to obtain CT image data of affected teeth and jaw bone data of a patient;
a parameter calculating module, configured to calculate calculation parameters of the surgery path based on the CT image data of the affected teeth obtained by the data obtaining module;
a path generating module, configured to obtain a start point and an ending point of the surgery path based on the calculation parameters of the surgery path and the jaw bone data to generate the surgery path. The data obtaining module, the parameter calculating module, and the path generating module may be realized as electronic circuits.

In one embodiment, there is provided an electronic device, which includes but not limited to various personal computers, laptop computers, smart phones and tablet computers and the like. The electronic device includes a processor, a memory, a network interface, a display screen, an input apparatus and a database connected via a system bus. The processor of the electronic device is configured to provide computation and control capabilities. The memory of the electronic device includes a nonvolatile storage medium and a memory. The nonvolatile storage medium stores an operating system, a computer program and a database. The memory provides an environment for the running of the operating system and the computer program in the nonvolatile storage medium. The database of the electronic device is configured to store the CT data of the affected teeth and the jaw bone data required for the apical surgery. The network interface of the electronic device is configured to communicate with an external terminal via network connection. The display screen of the electronic device may be a liquid crystal display screen or electronic ink display screen. The input apparatus of the electronic device may be a touch layer covered on the display screen or keys set on the housing of the electronic device or external keyboard, or touchpad or mouse or the like. The computer program may be executed by a processor to perform the method of the under-navigation apical surgery path.

There is provided a computer readable storage medium, storing computer programs thereon, where the computer programs are executed by a processor to perform the steps of the above method.

The above descriptions are about the preferred embodiments of the present disclosure and shall not be intended to limit the embodiments and the scope of protection of the present disclosure. Those skilled in the arts should understand that the solutions obtained by making equivalent replacements and obvious changes based on the contents of the specification of the present disclosure shall be incorporated in the scope of protection of the present disclosure.

1. A determination method of an under-navigation apical surgery fenestration path, comprising the following steps:
at step S1, obtaining CT image data of affected teeth and jaw bone data of a patient, and calculating the CT image data of the affected teeth to obtain calculation parameters of a surgery path;
at step S2, based on the calculation parameters of the surgery path and in combination with the data of the affected teeth and the jaw bone data, obtaining a start point and an ending point of the surgery path, and generating the surgery path based on the start point and the ending point;
wherein the calculation parameters of the step S1 comprise a dental root tip point root_tip and a direction root_vec of a dental root long axis;
the method of obtaining the calculation parameters comprises:
at step S101, obtaining three-dimensional model data of the affected teeth based on the CT image of the affected teeth to obtain a three-dimensional model of the affected teeth, and by using computer graphics algorithm, obtaining a center root_center of polydata of a dental root in the three-dimensional model of the affected teeth, wherein polydata refers to three-dimensional model data of the dental root;
at step S102, taking a center root_center of the dental root as origin and taking a direction of a dental long axis as normal direction, making a root-end resection plane plane_cut, wherein the direction of the dental long axis refers to pointing from a dental crown toward a dental root;
at step S103, by using the root-end resection plane plane_cut, cutting the three-dimensional model of the affected teeth into a first part part1 and a second part part2, using the first part part1 to calculate a connected region to obtain n connected regions, and extracting root polydata of a single connected region, wherein root_polydata refers to root three-dimensional data of a single connected region; and calculating the dental root tip point root_tip based on the root three-dimensional data root_polydata of the single connected region;
wherein the first part part1 is a positive direction part of the root-end resection plane plane_cut, and the second part part2 is a negative direction part of the root-end resection plane plane_cut; the dental root tip point root_tip is an angular point closest to a long axis origin in all angular points on the root polydata projected to the dental long axis;
at step S104, calculating a length left_length of a line connecting an origin orgin_cut on the root-end resection plane plane_cut with the dental root tip point root_tip, and determining whether the length left_length of the connection line exceeds a threshold; when the length left_length of the connection line exceeds the threshold, step S105 is performed; when the length left_length does not exceed the threshold, step S106 is performed;
at step S105, using a new origin obtained by translating the origin orgin_cut by a distance along the direction of the dental long axis and using the direction of the dental long axis as a normal direction, making the root-end resection plane plane_cut again, and repeating the steps S103 to S104 to perform multiple iterations on the single connected regions to extract the most connected regions until the length left_length is not beyond the threshold;
at step S106, obtaining a second root-end resection plane by translating the root-end resection plane by a distance along the normal direction, and using the second root-end resection plane to cut the root_polydata into a first part part1′ and a second part part2′ again, wherein the first part part1′ is a positive direction part of the second root-end resection plane and the second part part2′ is a negative direction part of the second root-end resection plane;
at step S107, using a center of the second part part2′ as a dental root top center root_top, using a line connecting the dental root tip point root_tip and the top center root_top as the direction root_vec of the dental root long axis, and using the dental root tip point root_tip and the direction root_vec of the dental root long axis as calculation parameters of the fenestration path;
the method of performing the step S2 includes:
at step S201, moving the dental root tip point root_tip along the direction root vec of the dental root long axis by an apicectomy distance to obtain a first calibration point Point1;
at step S202, making a first line segment Line1 through the first calibration point Point1 along a negative direction of a dental buccolingual direction Vec_coronal, and dividing intersection points of the first line segment Line1 and the three-dimensional model data polydata of the dental root into penetration-in points and penetration-out points;
wherein the penetration-in points and the penetration-out points are obtained by ranking the intersection points of the first line segment Line 1 and the three-dimensional model data polydata of the dental root, wherein the odd-numbered points are penetration-in points and the even-numbered points are penetration-out points;
at step S203, finding the penetration-in point Point_tmp and the penetration-out point Point_end which are closest to the dental root tip point root_tip;
at step S204, forming intersection points obtained by the first line segment Line1 and the jaw bone data; if the intersection points exist, taking a point among the intersection points, which is the closest point to a start point of the first line segment, as a start point Point_start of the path, and otherwise, taking the penetration-in point Point_tmp as the start point Point_start of the path.

2. The determination method of the under-navigation apical surgery fenestration path according to claim 1, wherein by using computer graphics algorithm, obtaining the center root_center of the polydata of the dental root comprises: by using computer graphics algorithm, segmenting the three-dimensional model of the affected teeth into a three-dimensional model of the dental crown and a three-dimensional model of the dental root, and calculating a centroid of the three-dimensional model of the dental root to obtain the center root_cente of the polydata of the dental root.

3. A determination apparatus for performing the under-navigation apical surgery fenestration path according to claim 1, comprising:
a data obtaining module, configured to obtain CT image data of affected teeth and jaw bone data of a patient;
a parameter calculating module, configured to calculate calculation parameters of the surgery path based on the CT image data of the affected teeth obtained by the data obtaining module;
a path generating module, configured to obtain a start point and an ending point of the surgery path based on the calculation parameters of the surgery path and the jaw bone data to generate the surgery path.

4. An electronic device, comprising a memory and a processor, wherein the memory stores computer programs, wherein the processor executes the computer programs to perform the steps of the determination method of claim 1.

5. A computer readable storage medium, storing computer programs, wherein the computer programs are executed by a processor to perform the steps of the determination method of claim 1.

6. A determination apparatus for performing the under-navigation apical surgery fenestration path according to claim 2, comprising:
a data obtaining module, configured to obtain CT image data of affected teeth and jaw bone data of a patient;
a parameter calculating module, configured to calculate calculation parameters of the surgery path based on the CT image data of the affected teeth obtained by the data obtaining module;
a path generating module, configured to obtain a start point and an ending point of the surgery path based on the calculation parameters of the surgery path and the jaw bone data to generate the surgery path.

7. An electronic device, comprising a memory and a processor, wherein the memory stores computer programs, wherein the processor executes the computer programs to perform the steps of the determination method of claim 2.

8. A computer readable storage medium, storing computer programs, wherein the computer programs are executed by a processor to perform the steps of the determination method of claim 2.
